(19) United States
(12) Patent Application Publication
(10) Pub. No.: US 2026/0233383 A1
(43) Pub. Date: Aug. 13, 2026

(54) SYSTEM FOR CONTROLLING CONTINUUM ROBOT, AND METHOD FOR CONTROLLING CONTINUUM ROBOT

(71) Applicant: CANON KABUSHIKI KAISHA, Tokyo (JP)

(72) Inventor: KIYOSHI TAKAGI, Tokyo (JP)

(21) Appl. No.: 19/397,467

(22) Filed: Nov. 21, 2025

Related U.S. Application Data
(63) Continuation of application No. PCT/JP2024/018252, filed on May 17, 2024.

(30) Foreign Application Priority Data
May 24, 2023 (JP) 2023-085690

(51) Int. Cl. B25J 9/10; B25J 9/06; B25J 13/08; B25J 18/06

(52) U.S. Cl. CPC B25J 9/1045; B25J 9/065; B25J 13/085; B25J 18/06

(57) ABSTRACT

A system for controlling a continuum robot, the continuum robot including a bendable portion and a wire driving unit, the bendable portion including a plurality of bending sections each driven by a wire, the wire driving unit being configured to drive the wire for each of the plurality of bending sections, includes a position control unit, a generated force calculation unit, and a force control unit that are a control unit configured to control operation of the wire driving unit for each of the plurality of bending sections, and a coupling control unit that is one component of a suppression unit configured to, when the wire driving unit for one of the plurality of bending sections is operated, suppress variation in tension of the wires for other bending sections.

### CROSS-REFERENCE TO RELATED APPLICATIONS

This application is a Continuation of International Patent Application No. PCT/JP2024/018252, filed May 17, 2024, which claims the benefit of Japanese Patent Application No. 2023-085690, filed May 24, 2023, both of which are hereby incorporated by reference herein in their entirety.

### BACKGROUND

### Field of the Technology

The present disclosure relates to a system for controlling a continuum robot and a method for controlling a continuum robot.

### Description of the Related Art

A continuum robot includes a bendable portion including a plurality of bending sections with a flexible structure, and shape of the continuum robot is controlled by deforming the bendable portion. The continuum robot has two main advantages over a robot composed of rigid links (hereinafter referred to as a “rigid-link robot”). The first advantage is that the continuum robot can move along curves of an object in narrow spaces or environments with scattered objects where the rigid-link robot may become stuck. The second advantage is that, because the continuum robot inherently has softness, the continuum robot can perform operation without causing damage to a fragile object. In such cases, detection of external forces that is required for the rigid-link robot is not necessarily required.

Taking advantage of this feature, the continuum robot is expected to be applied to a medical field, for example, an endoscope sheath and a catheter, and to a robot for extreme environments, such as a rescue robot. Furthermore, when a driving unit passively operates based on a load, this is referred to as having backdrivability, and there is a continuum robot that has high backdrivability through a mechanism and a control algorithm.

Japanese Patent Laid-Open No. 2021-133429 describes a dynamic model of a continuum robot including a wire driving unit that can detect tension of a wire that bends a bendable portion through driving. Further, in Japanese Patent Laid-Open No. 2021-133429, a dual-loop control system is implemented in which an inner loop control system performs force control on the continuum robot and an outer loop control system performs position control on the continuum robot. Accordingly, Japanese Patent Laid-Open No. 2021-133429 realizes the continuum robot that has high backdrivability against disturbances at a tip of the continuum robot and enables positioning to a target position.

In Japanese Patent Laid-Open No. 2021-133429, stability of a feedback system is ensured in a force control system and a position control system by using a transfer function obtained from the dynamic model of the continuum robot. In this case, in Japanese Patent Laid-Open No. 2021-133429, a continuum robot including a single bending section is used as a model. Furthermore, in Japanese Patent Laid-Open No. 2021-133429, when the technique is applied to a continuum robot including a plurality of bending sections, a distributed backdrivable control system in which the above-described control systems are applied is used for each of the bending sections.

However, in the continuum robot including the bendable portion including the plurality of bending sections, coupling between the bending sections occurs. In the coupling, motion of one bending section affects motion of another bending section. Since Japanese Patent Laid-Open No. 2021-133429 does not take the coupling between the bending sections into consideration, there is an issue that stability of the control systems of the continuum robot decreases due to a change in mechanism, such as a change in the shape of the continuum robot and an increase or decrease in friction elements.

### SUMMARY

The present disclosure is made in consideration of such an issue, and is directed to a technique that can suppress a decrease in stability of a control system of a continuum robot.

According to an aspect of the present disclosure, a system for controlling a continuum robot, the continuum robot including a bendable portion and a wire driving unit, the bendable portion including a plurality of bending sections each driven by a wire, the wire driving unit being configured to drive the wire for each of the plurality of bending sections, includes a control unit configured to control operation of the wire driving unit for each of the plurality of bending sections, and a suppression unit configured to, when the wire driving unit for one of the plurality of bending sections is operated, suppress variation in tension of the wires for other bending sections.

Features of the present disclosure will become apparent from the following description of embodiments with reference to the attached drawings.

### BRIEF DESCRIPTION OF THE DRAWINGS

FIG. 1 is a diagram schematically illustrating an example of a configuration of a continuum robot according to a first embodiment.

FIG. 2A is a diagram schematically illustrating an example of a configuration of a system for controlling the continuum robot according to the first embodiment.

FIG. 2B is a diagram schematically illustrating an example of the configuration of the system for controlling the continuum robot according to the first embodiment.

FIG. 3 is a diagram schematically illustrating an example of a configuration of a continuum robot according to a second embodiment.

FIG. 4A is a diagram schematically illustrating an example of a configuration of a continuum robot according to a third embodiment.

FIG. 4B is a diagram schematically illustrating an example of a configuration of the continuum robot according to the third embodiment.

FIG. 5 is a diagram illustrating an example of a dynamic model of the continuum robot (continuum portion) according to the third embodiment.

FIG. 6A is a diagram illustrating an example of a model including a rotary motor, a rotary-to-linear conversion mechanism, and a wire holding mechanism in the continuum robot according to the third embodiment.

FIG. 6B is a diagram illustrating a configuration example in which the dynamic model of the continuum portion illustrated in FIG. 5 and the model including the rotary motor, the rotary-to-linear conversion mechanism, and the wire holding mechanism illustrated in FIG. 6A are joined in the continuum robot according to the third embodiment.

FIG. 7A is a diagram illustrating an example of a frequency response when c_{w21}=c_{w22}=0 Ns/m is set in the continuum robot according to the third embodiment.

FIG. 7B is a diagram illustrating an example of the frequency response when c_{w21}=c_{w22}=0 Ns/m is set in the continuum robot according to the third embodiment.

FIG. 8A is a diagram illustrating an example of a frequency response when c_{w21}=c_{w22}=150 Ns/m is set in the continuum robot according to the third embodiment.

FIG. 8B is a diagram illustrating an example of the frequency response when c_{w21}=c_{w22}=150 Ns/m is set in the continuum robot according to the third embodiment.

FIG. 9 is a Bode diagram of a force control unit in a case where a gain Kₚ is set to 2.2·10⁻³, a zero-cross frequency F_{zi} is set to 1.0, and a control bandwidth is set to approximately 10 Hz in a system for controlling a continuum robot according to a fourth embodiment.

FIG. 10 is a diagram obtained by plotting variation of eigenvalues of a closed-loop system in the system for controlling the continuum robot according to the fourth embodiment.

FIG. 11A is a diagram illustrating an example of a frequency response of the continuum robot in a case where quasi-optimal damping coefficients of coupling control dampers are set to c_{w21}=c_{w22}=25 Ns/m in the system for controlling the continuum robot according to the fourth embodiment.

FIG. 11B is a diagram illustrating an example of the frequency response of the continuum robot in the case where quasi-optimal damping coefficients of the coupling control dampers are set to c_{w21}=c_{w22}=25 Ns/m in the system for controlling the continuum robot according to the fourth embodiment.

FIG. 12 is a Bode diagram of a position control unit in a case where the gain Kₚ is set to 5·10³, the zero-cross frequency F_{zi} is set to 0.5, and a zero-cross frequency F_{zd} is set to 100 in the system for controlling the continuum robot according to the fourth embodiment.

FIG. 13A is a diagram illustrating simulation responses in the system for controlling the continuum robot according to the fourth embodiment, where a quasi-optimal model is illustrated with a solid line, a low damping model is illustrated with a dashed line, and a high damping model is illustrated with a dash-dot line.

FIG. 13B is a diagram illustrating simulation responses in the system for controlling the continuum robot according to the fourth embodiment, where the quasi-optimal model is illustrated with a solid line, the low damping model is illustrated with a dashed line, and the high damping model is illustrated with a dash-dot line.

FIG. 13C is a diagram illustrating simulation responses in the system for controlling the continuum robot according to the fourth embodiment, where the quasi-optimal model is illustrated with a solid line, the low damping model is illustrated with a dashed line, and the high damping model is illustrated with a dash-dot line.

FIG. 13D is a diagram illustrating simulation responses in the system for controlling the continuum robot according to the fourth embodiment, where the quasi-optimal model is illustrated with a solid line, the low damping model is illustrated with a dashed line, and the high damping model is illustrated with a dash-dot line.

FIG. 13E is a diagram illustrating simulation responses in the system for controlling the continuum robot according to the fourth embodiment, where the quasi-optimal model is illustrated with a solid line, the low damping model is illustrated with a dashed line, and the high damping model is illustrated with a dash-dot line.

FIG. 13F is a diagram illustrating simulation responses in the system for controlling the continuum robot according to the fourth embodiment, where the quasi-optimal model is illustrated with a solid line, the low damping model is illustrated with a dashed line, and the high damping model is illustrated with a dash-dot line.

### DESCRIPTION OF THE EMBODIMENTS

Embodiments of the present disclosure are described below with reference to the drawings.

### First Embodiment

First, a first embodiment is described.

### [1] Configuration of Continuum Robot

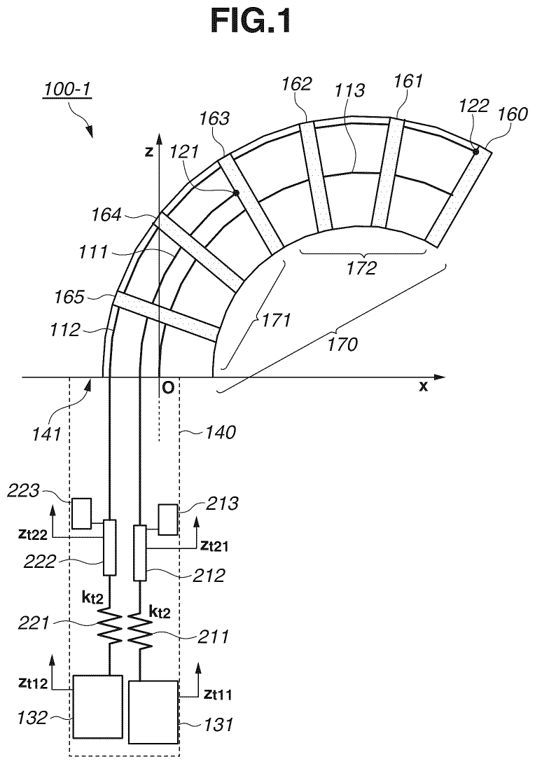

FIG. 1 is a diagram illustrating an example of a schematic configuration of a continuum robot 100 according to the first embodiment. In the following description, the continuum robot 100 according to the first embodiment illustrated in FIG. 1 is referred to as a “continuum robot 100-1”.

As illustrated in FIG. 1, the continuum robot 100-1 includes a base unit 140 and a bendable portion 170. The bendable portion 170 is a component including a plurality of bending sections 171 and 172 (the component including plurality of bending sections) that are bent when wires are driven. The base unit 140 is a component supporting the bendable portion 170, and inside the base unit 140, there are provided actuators 131 and 132 that are wire driving units for respectively driving wires 111 and 112 of the bending sections 171 and 172. FIG. 1 illustrates an example in which, as the plurality of bending sections provided in the bendable portion 170, two bending sections, namely the first bending section 171 and the second bending section 172 are provided; however, in the present embodiment, the plurality of bending sections is not limited thereto. For example, a form in which, as the plurality of bending sections provided in the bendable portion 170, three or more bending sections are provided is also applicable to the present embodiment.

In FIG. 1, an origin O is set at a predetermined position on an upper surface 141 of the base unit 140. Further, FIG. 1 illustrates an xz coordinate system in which, for example, an advancing direction of the continuum robot 100-1 is defined as a z direction of the coordinate system and a predetermined direction orthogonal to the z direction is defined as an x direction of the coordinate system.

The wire 111 is connected to a connection portion 121 at a distal end 163 of the first bending section 171. An attitude (bent shape) of the continuum robot 100-1 is controlled by the wire 111 being pushed and pulled by the actuator 131 installed inside the base unit 140. Here, the actuator 131 is the wire driving unit for driving the wire 111.

The wire 112 is connected to a connection portion 122 at a distal end 160 of the second bending section 172. The attitude (bent shape) of the continuum robot 100-1 is controlled by the wire 112 being pushed and pulled by the actuator 132 installed inside the base unit 140. Here, the actuator 132 is the wire driving unit for driving the wire 112.

The continuum robot 100-1 includes wire guides 164 and 165 that are members for guiding the wire 111 and the wire 112 in the first bending section 171. The continuum robot 100-1 further includes wire guides 161 and 162 that are members for guiding the wire 112 in the second bending section 172. The distal ends and wire guides 160 to 165 are fixed to a backbone 113. The continuum robot 100-1 has a configuration in which a plurality of members including the distal ends and wire guides 160 to 165 is discretely arranged, but may also be configured using a continuum member having a bellows shape, a mesh shape, or another shape in addition thereto.

In an internal mechanism of the base unit 140 for the first bending section 171 driven by the wire 111, a wire holding unit 212 holding the wire 111 and a spring 211 are interposed between the wire 111 and the actuator 131. Similarly, in the internal mechanism of the base unit 140 for the second bending section 172 driven by the wire 112, a wire holding unit 222 holding the wire 112 and a spring 221 are interposed between the wire 112 and the actuator 132.

A displacement zₜ₁₁ of the actuator 131 and a displacement zₜ₂₁ of the wire holding unit 212 for the first bending section 171 can be detected by an encoder or the like. Based on these displacements and a spring constant kₜ₂ of the spring 211, wire tension of the wire 111 for driving (bending) the first bending section 171 can be detected. At this time, the spring 211 is one component of a tension detection unit for detecting the tension of the wire 111. Similarly, based on a displacement zₜ₁₂ of the actuator 132, a displacement zₜ₂₂ of the wire holding unit 222 for the second bending section 172, and a spring constant kₜ₂ of the spring 221, wire tension of the wire 112 for driving (bending) the second bending section 172 can be detected. At this time, the spring 221 is one component of a tension detection unit for detecting the tension of the wire 112.

Further, in the present embodiment, in the internal mechanism of the base unit 140 for the first bending section 171 driven by the wire 111, a coupling control actuator 213 is connected to the wire holding unit 212. In the coupling control actuator 213, a surface on a side opposite the wire 111 side is connected to the base unit 140. Similarly, in the present embodiment, in the internal mechanism of the base unit 140 for the second bending section 172 driven by the wire 112, a coupling control actuator 223 is connected to the wire holding unit 222.

In the coupling control actuator 223, a surface on a side opposite the wire 112 side is connected to the base unit 140.

Next, coupling between the first bending section 171 and the second bending section 172 is described.

For example, as illustrated in FIG. 1, when the actuator 132 operates in a positive direction, the second bending section 172 is bent. However, at the same time, the first bending section 171 is bent because torque is applied to the distal end 163 of the first bending section 171 via members such as the backbone 113. When position control is applied to each of the first bending section 171 and the second bending section 172, the first bending section 171 and the second bending section 172 can be bent into any desired shape, but the wire tension of the wire 111 or the wire 112 is superimposed with a part of the wire tension of the other bending section. In other words, when the actuator 132 operates, the tension of the wire 111 is changed in addition to the tension of the wire 112. Similarly, when the actuator 131 operates, the tension of the wire 112 is changed in addition to the tension of the wire 111. In the present embodiment, this is defined as coupling between the bending sections. When a control system is designed in consideration of wire tension of a single bending section as described in Japanese Patent Laid-Open No. 2021-133429 without taking the coupling between the bending sections into consideration, and the control system is applied to a continuum robot including a plurality of bending sections, an issue that stability of the control system is changed (decreased) arises. In particular, when frictional force applied to the driven wire is reduced, the coupling between the bending sections is increased, and the control system becomes unstable. In the present embodiment, change in the tension of the wire 111 when the actuator 131 is driven and change in the tension of the wire 112 when the actuator 132 is driven are termed as direct characteristics.

### [2] Configuration of Control System

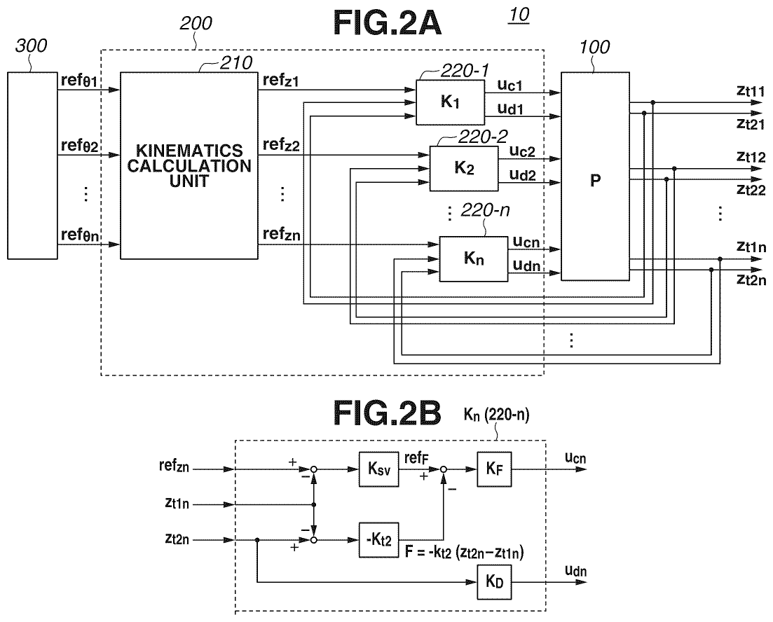

FIGS. 2A and 2B are diagrams illustrating an example of a schematic configuration of a system 10 for controlling the continuum robot according to the first embodiment. As illustrated in FIG. 2A, the system 10 for controlling the continuum robot includes, for example, a mechanism unit P of the continuum robot 100-1 (hereinafter simply referred to as “continuum robot 100”) illustrated in FIG. 1, a continuum robot control apparatus 200, and an input apparatus 300.

As illustrated in FIG. 2A, the continuum robot control apparatus 200 includes a kinematics calculation unit (kinematics) 210, and controllers 220-1 to 220-n. The controller 220-1 is, for example, a controller corresponding to the first bending section 171 illustrated in FIG. 1. The controller 220-2 is, for example, a controller corresponding to the second bending section 172 illustrated in FIG. 1. Further, the controller 220-n is, for example, in a case where three or more bending sections are configured in the continuum robot 100, a controller corresponding to a certain bending section among the three or more bending sections. Further, as illustrated in FIG. 2B, the controller 220-n illustrated in FIG. 2A includes a position control unit K_{SV}, a force control unit K_{F}, a generated force calculation unit −kₜ₂, and a coupling control unit K_{D}, for example. While FIG. 2B illustrates an internal configuration of the controller 220-n illustrated in FIG. 2A, each of the controller 220-1 and the controller 220-2 illustrated in FIG. 2A also has an internal configuration similar to the internal configuration of the controller 220-n illustrated in FIG. 2B. In this case, for the internal configurations of the controller 220-1 and the controller 220-2 illustrated in FIG. 2A, a form in which the reference numeral “n” of the controller 220-n illustrated in FIG. 2B is changed to the reference numerals “1” and “2” is adopted. Hereinafter, in a case where matters common to the controllers 220-1 to 220-n are described without specifying the controllers 220-1 to 220-n, the controller is simply referred to as a “controller 220”.

In the present embodiment, in the controller 220 illustrated in FIG. 2B, an inner loop control system (first loop control system) including the force control unit K_{F} performs force control on the continuum robot 100. Further, in the controller 220 illustrated in FIG. 2B, an outer loop control system (second loop control system) including the force control unit K_{F} and the position control unit K_{SV} performs position control on the continuum robot 100. The system 10 for controlling the continuum robot according to the present embodiment includes a dual-loop control system of the inner loop control system (first loop control system) and the outer loop control system (second loop control system). In the system 10 for controlling the continuum robot according to the present embodiment, with the dual-loop control system, high backdrivability for the continuum robot 100 is achieved. At the same time, high positioning performance for a target position of the bendable portion 170 of the continuum robot 100 is achieved.

Further, in the present embodiment, the controller 220 illustrated in FIG. 2B includes the coupling control unit K_{D} to improve stability of the control system (to suppress a decrease in stability) caused by the above-described coupling between the bending sections. For example, a coupling control unit K_{D} of the controller 220-1 provides a control input to the coupling control actuator 213 to suppress a decrease in stability of the control system caused by the above-described coupling between the bending sections. Further, for example, a coupling control unit K_{D} of the controller 220-2 provides a control input to the coupling control actuator 223 to suppress a decrease in stability of the control system caused by the above-described coupling between the bending sections.

Next, a specific flow of signals in the system 10 for controlling the continuum robot is described.

When receiving target bending angles ref_{θ1} to ref_{θn} for the respective bending sections of the continuum robot 100 from the input apparatus 300, the kinematics calculation unit 210 illustrated in FIG. 2A performs kinematics calculation. Thereafter, the kinematics calculation unit 210 outputs target displacements ref_{z1} to ref_{zn} of push-pull driving for the wires 111, 112, . . . by the actuators 131, 132, . . . of the continuum robot 100, to the respective controllers 220-1 to 220-n.

The position control unit K_{SV} illustrated in FIG. 2B is a unit that performs control to compensate an error between a target displacement ref_{z} output from the kinematics calculation unit 210 and a displacement zₜ₁ of the actuator, and outputs a target generated force ref_{F} corresponding to target tension of the wire. The target generated force ref_{F} indicates a target value of a generated force F that is a positive/negative inversion value of a wire tension sensor as defined in the present embodiment.

The generated force calculation unit −kₜ₂ illustrated in FIG. 2B outputs the generated force F corresponding to the tension of the wire based on a difference between a displacement zₜ₂ of the wire holding unit and a displacement zₜ₁ of the actuator, and the spring constant kₜ₂ of the spring. The force control unit K_{F} illustrated in FIG. 2B is a unit that performs control to compensate an error between the target generated force ref_{F} output from the position control unit K_{SV} and the generated force F output from the generated force calculation unit −kₜ₂, and outputs a target torque uc for the actuator that is the wire driving unit.

A feedback loop control system in the continuum robot control apparatus 200 illustrated in FIG. 2A is equivalent to compensating for equivalent inertia of a rotary-to-linear conversion mechanism, thereby making it possible to improve the backdrivability of the continuum robot 100.

Further, in the present embodiment, the controller 220 illustrated in FIG. 2B includes a third loop control system that is independent of the above-described first and second loop control systems and includes the coupling control unit K_{D} that controls operation of the coupling control actuator based on the displacement zₜ₂ of the wire holding unit. In the third loop control system, the coupling control unit K_{D} receives the displacement z12 of the wire holding unit as an input signal, and provides a control input u_{d} to the coupling control actuator to suppress the displacement zₜ₂ of the wire holding unit.

For example, when the third loop control system uses direct speed control using the coupling control unit K_{D} as a differentiator, the third loop control system constitutes a direct speed feedback control system. In this case, the coupling control actuators 213 and 223 function as dampers. When the actuator 132 operates, the third loop control system suppresses variation of the tension of the coupled wire 111, whereas when the actuator 131 operates, the third loop control system suppresses variation of the tension of the coupled wire 112. As described above, by the third loop control system being provided in the controller 220, coupling between the bending sections is suppressed, and the suitable first and second loop control systems are designed in consideration of only the direct characteristics of each of the bending sections. Further, the coupling control unit K_{D} may be a control system in which a lowpass filter is connected in series with the differentiator, or may be a proportional-derivative (PD) control system or a proportional-integral-derivative (PID) control system. The coupling control unit K_{D} may be designed by evaluating stability of the first and second loop control systems. For the evaluation at this time, a numerical model of the continuum robot 100 may be used, or an experimental system may be directly measured.

The system 10 for controlling the continuum robot 100 according to the first embodiment described above includes the following configuration. First, the system 10 includes the continuum robot 100 that includes the bendable portion 170 and the actuators 131 and 132. The bendable portion 170 includes the plurality of bending sections 171 and 172. The actuators 131 and 132 are the wire driving units that drive the wires 111 and 112 for the respective bending sections. The system 10 further includes the position control units K_{SV}, the generated force calculation units −kₜ₂, and the force control units K_{F} that are control units that control operation of the actuators 131 and 132 that are the wire driving units for the respective bending sections. The system 10 further includes suppression units each suppressing, when the actuator 131 or 132 that is the wire driving unit in the corresponding bending section operates, variation of the tension of the wire 112 or 111 in the other bending section. More specifically, the suppression unit for the first bending section 171 includes the coupling control actuator 213 that is a second wire driving unit, and the coupling control unit K_{D} for the first bending section 171 that is a second control unit that controls operation of the coupling control actuator 213. At this time, the coupling control actuator 213 for the first bending section 171 is connected to the wire holding unit 212 that holds the wire 111 so as to drive the wire 111 without interposing the spring 211 that is the tension detection unit. Further, the coupling control unit K_{D} for the first bending section 171 controls operation of the coupling control actuator 213 based on the displacement z121 of the wire holding unit 212 that is the wire holding unit. Similarly, for example, the suppression unit for the second bending section 172 is a unit that suppresses, when the actuator 132 that is the wire driving unit operates, variation of the tension of the wire 111 in the other bending section 171. More specifically, the suppression unit for the second bending section 172 includes the coupling control actuator 223 that is the second wire driving unit, and the coupling control unit K_{D} for the second bending section 172 that is the second control unit that controls operation of the coupling control actuator 223. At this time, the coupling control actuator 223 for the second bending section 172 is connected to the wire holding unit 212 that holds the wire 112 so as to drive the wire 112 without interposing the spring 221 that is the tension detection unit. Further, the coupling control unit K_{D} for the second bending section 172 controls operation of the coupling control actuator 223 based on the displacement zₜ₂₂ of the wire holding unit 222 that is the wire holding unit.

Since such a configuration includes the suppression unit, it is possible to reduce influence of operation of a certain bending section to be exerted on operation of the other bending section, and to suppress mutual coupling between the bending sections.

Therefore, in the continuum robot 100 including the plurality of bending sections, it is possible to suppress a decrease in stability of the control unit (control system) that controls operation of the corresponding bending section for each bending section among the plurality of bending sections.

In the present embodiment, the displacement zₜ₂₁ and the displacement zₜ₂₂ on which the control of the coupling control unit K_{D} is based are the displacements of the wire holding unit 212 and the wire holding unit 222. However, the displacements are not limited to the displacements of the wire holding units as long as the displacements are displacements of a portion between a connection portion of the coupling control actuator 213 and the spring 211 that is the tension detection unit, and a portion between a connection portion of the coupling control actuator 223 and the spring 221 that is the tension detection unit.

Further, although the example in which the coupling control actuator is connected to the wire holding unit is described, an object to which the coupling control actuator is connected may be replaced with any unit that connects the wire and the tension detection unit instead of the wire holding unit.

### Second Embodiment

Next, a second embodiment is described. In the following description of the second embodiment, description of matters common to the above-described first embodiment is omitted, and matters different from the above-described first embodiment are described.

In the system 10 for controlling the continuum robot according to the above-described first embodiment, as the suppression unit for suppressing the coupling between the bending sections, the coupling control actuators 213 and 223 that are the second wire driving units, and the coupling control unit K_{D} that is a second coupling unit are provided. In contrast, in a system 10 for controlling a continuum robot according to the second embodiment, as the suppression unit for suppressing coupling between the bending sections, coupling control dampers connected to the respective wire holding units 212 and 222 are provided in the continuum robot 100.

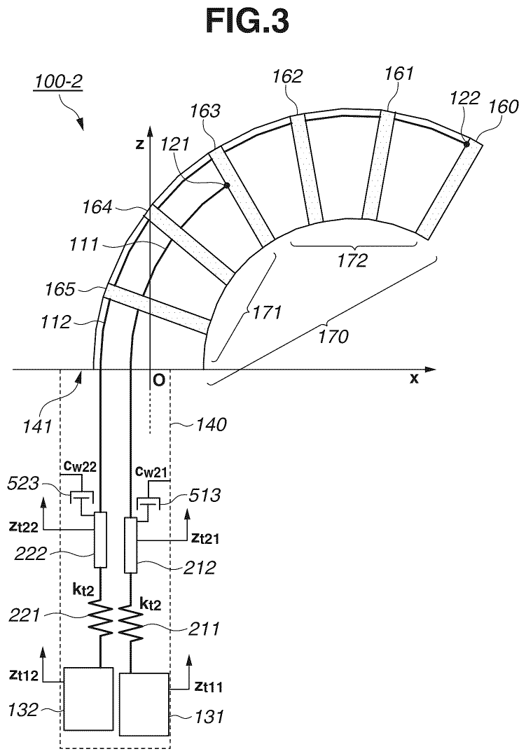

FIG. 3 is a diagram schematically illustrating an example of a configuration of the continuum robot 100 according to the second embodiment. In the following description, the continuum robot 100 according to the second embodiment illustrated in FIG. 3 is referred to as a “continuum robot 100-2”. In FIG. 3, components similar to the components illustrated in FIG. 1 are denoted by the same reference numerals, and detailed description of the components is omitted.

In the continuum robot 100-2 illustrated in FIG. 3, as the suppression unit for the first bending section 171, a coupling control damper 513 connected to the wire holding unit 212 is provided. A side of the coupling control damper 513 opposite a wire holding unit 212 side is connected to the base unit 140. Further, in the continuum robot 100-2 illustrated in FIG. 3, as the suppression unit for the second bending section 172, a coupling control damper 523 connected to the wire holding unit 222 is provided. A side of the coupling control damper 523 opposite a wire holding unit 222 side is connected to the base unit 140. These coupling control dampers 513 and 523 are equivalent to the configuration where a differentiator is used for the coupling control unit K_{D} in the first embodiment. At this time, damping coefficients of the coupling control dampers 513 and 523 may be directly measured and set, or may be set using a numerical model (dynamic model) of the continuum robot 100-2.

In the second embodiment, as in the first embodiment, in the continuum robot 100 including the plurality of bending sections, it is possible to suppress a decrease in stability of the control unit (control system) that controls operation of the corresponding bending section for each bending section among the plurality of bending sections.

### Third Embodiment

Next, a third embodiment is described. In the following description of the third embodiment, description of matters common to the above-described first and second embodiments is omitted, and matters different from the above-described first and second embodiments are described.

In the system 10 for controlling the continuum robot according to the above-described first embodiment, the coupling control actuators 213 and 223 are respectively provided in the wire holding units 212 and 222. In the system 10 for controlling the continuum robot according to the above-described second embodiment, the coupling control dampers 513 and 523 are respectively provided in the wire holding units 212 and 222. In contrast, in a system 10 for controlling a continuum robot 100 according to the third embodiment, a coupling control actuator or a coupling control damper is provided for a wire.

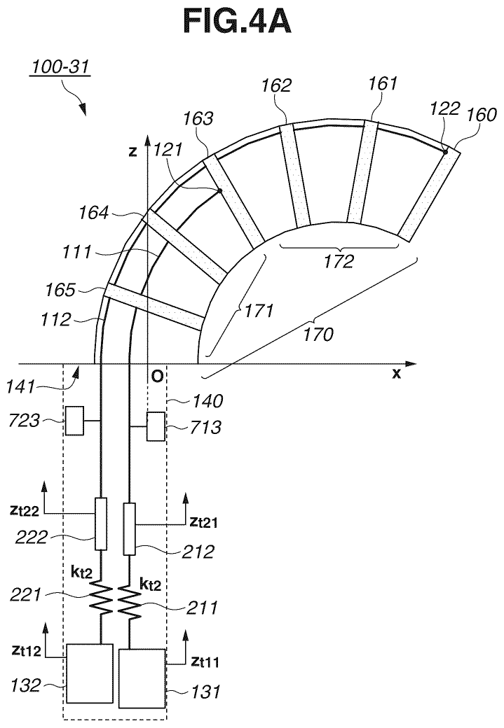

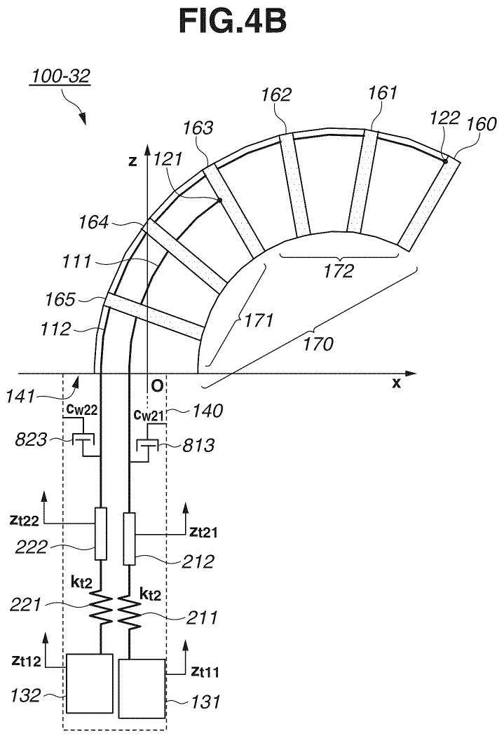

FIGS. 4A and 4B are diagrams each schematically illustrating an example of a configuration of the continuum robot 100 according to the third embodiment. In the following description, the continuum robot 100 according to a first example of the third embodiment illustrated in FIG. 4A is described as a “continuum robot 100-31”, and the continuum robot 100 according to a second example of the third embodiment illustrated in FIG. 4B is described as a “continuum robot 100-32”.

Further, in FIG. 4A and FIG. 4B, components similar to the components illustrated in FIG. 1 and FIG. 3 are denoted by the same reference numerals, and detailed description of the components is omitted.

In the continuum robot 100-31 illustrated in FIG. 4A, a coupling control actuator 713 is provided for the wire 111 in the first bending section 171, and a coupling control actuator 723 is provided for the wire 112 in the second bending section 172. In this configuration, the coupling control actuator 713 is provided in place of the coupling control actuator 213 of the continuum robot 100-1 illustrated in FIG. 1, and the coupling control actuator 723 is provided in place of the coupling control actuator 223 of the continuum robot 100-1 illustrated in FIG. 1.

In the continuum robot 100-32 illustrated in FIG. 4B, a coupling control damper 813 is provided for the wire 111 in the first bending section 171, and a coupling control damper 823 is provided for the wire 112 in the second bending section 172. In this configuration, the coupling control damper 813 is provided in place of the coupling control damper 513 of the continuum robot 100-2 illustrated in FIG. 3, and the coupling control damper 823 is provided in place of the coupling control damper 523 of the continuum robot 100-2 illustrated in FIG. 3.

As in the above-described first embodiment, the coupling control unit K_{D} of each of the coupling control actuators 713 and 723 illustrated in FIG. 4A may be designed by evaluating stability of the first and second loop control systems. For the evaluation at this time, a numerical model of the continuum robot 100 may be used, or an experimental system may be directly measured. Further, in the present embodiment, damping coefficients of the coupling control dampers 813 and 823 illustrated in FIG. 4B are designed using a dynamic model of the continuum robot 100. Details thereof are described below. Further, in the present embodiment as well, the terms “first bending section 171” and “second bending section 172” are used.

### [1] Modeling of Continuum Robot 100

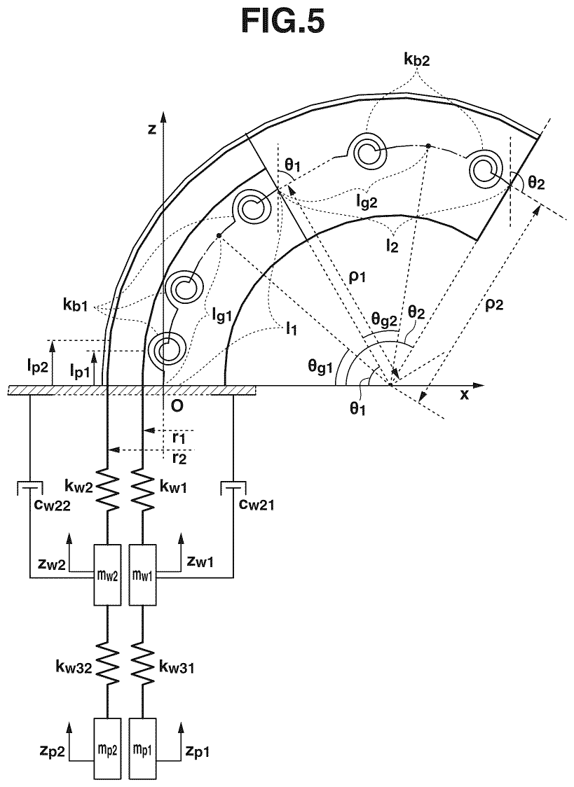

FIG. 5 is a diagram illustrating an example of the dynamic model of the continuum robot 100 (continuum portion) according to the third embodiment. The definitions of reference symbols for the dynamic model of the continuum robot 100 (continuum portion) illustrated in FIG. 5 are as follows.
θn: bending angle of the continuum robot
mₛₙ: mass of the continuum portion
k_{bn}: spring constant with respect to the bending angle of the continuum portion
z_{wn}: displacement of equivalent mass of a wire
zₚₙ: displacement of a wire holding pipe
m_{wn}: mass of the wire
mₚₙ: mass of the wire holding pipe
k_{wn}, k_{w3n}: spring constants of the wire
c_{wn}, c_{w3n}: damping coefficients of the wire
c_{w2n}: damping coefficient of the coupling control damper

Here, the number of bending sections n is set to 2 (the first bending section 171 and the second bending section 172), and equations of motion of the continuum robot 100 are derived. In the present embodiment, the following assumptions are made:
<1> Only motion in a two-dimensional plane (xz plane) is considered.
<2> Curvature of the continuum portion is constant, and spring constant is uniform.
<3> Wire is approximated as lumped mass system, and a reaction force caused by deformation in a longitudinal direction acts on a tip of the continuum portion. Lateral vibration and lateral deformation of the wire are not considered.
<4> Friction between the wire and the wire guide, and between the wire and a diameter conversion unit, including non-linear friction such as Coulomb friction, is incorporated into a damping coefficient c_{w2} as viscous damping.

First, kinetic energy of the continuum portion is obtained. For the first bending section 171, when displacements x_{g1} and z_{g1} are taken on a center axis of the continuum portion, the displacements x_{g1} and z_{g1} are expressed by the following equations (1) and (2), respectively.

$${x}_{g 1} = \frac{{l}_{g 1}}{{θ}_{g 1}} ( 1 - cos {θ}_{g 1} ) \qquad ( 1 )$$

$${z}_{g 1} = \frac{{l}_{g 1}}{{θ}_{g 1}} sin {θ}_{g 1} \qquad ( 2 )$$

Further, since the following equation (3) holds, the above-described equations (1) and (2) become the following equations (4) and (5), respectively.

$${θ}_{g 1} = \frac{{l}_{g 1}}{{l}_{1}} {θ}_{1} \qquad ( 3 )$$

$${x}_{g 1} = \frac{{l}_{1}}{{θ}_{1}} ( 1 - cos \frac{{l}_{g 1}}{{l}_{1}} {θ}_{1} ) \qquad ( 4 )$$

$${z}_{g 1} = \frac{{l}_{1}}{{θ}_{1}} sin \frac{{l}_{g 1}}{{l}_{1}} {θ}_{1} \qquad ( 5 )$$

For the second bending section 172, when displacements x_{g2} and z_{g2} are taken on the center axis of the continuum portion, the displacements x_{g2} and z_{g2} are expressed by the following equation (6).

$$[ \begin{matrix}{x}_{g 2} \\ {z}_{g 2}\end{matrix} ] = [ \begin{matrix}\frac{{l}_{1}}{{θ}_{1}} ( 1 - cos {θ}_{1} ) \\ \frac{{l}_{1}}{{θ}_{1}} sin {θ}_{1}\end{matrix} ] + [ \begin{matrix}cos {θ}_{1} & sin {θ}_{1} \\ - sin {θ}_{1} & cos {θ}_{1}\end{matrix} ] [ \begin{matrix}\frac{{l}_{g 2}}{{θ}_{g 2}} ( 1 - cos {θ}_{g 2} ) \\ \frac{{l}_{g 2}}{{θ}_{g 2}} sin {θ}_{g 2}\end{matrix} ] \qquad ( 6 )$$

Further, since the following equation (7) holds, the above-described equation (6) becomes the following equation (8).

$${θ}_{g 2} = \frac{{l}_{g 2}}{{l}_{2}} ( {θ}_{2} - {θ}_{1} ) \qquad ( 7 )$$

$$[ \begin{matrix}{x}_{g 2} \\ {z}_{g 2}\end{matrix} ] = [ \begin{matrix}\frac{{l}_{1}}{{θ}_{1}} ( 1 - cos {θ}_{1} ) \\ \frac{{l}_{1}}{{θ}_{1}} sin {θ}_{1}\end{matrix} ] + [ \begin{matrix}cos {θ}_{1} & sin {θ}_{1} \\ - sin {θ}_{1} & cos {θ}_{1}\end{matrix} ] [ \begin{matrix}\frac{{l}_{2}}{{θ}_{2} - {θ}_{1}} \{ 1 - cos ( \frac{{l}_{g 2}}{{l}_{2}} ( {θ}_{2} - {θ}_{1} ) ) \} \\ \frac{{l}_{2}}{{θ}_{2} - {θ}_{1}} sin ( \frac{{l}_{g 2}}{{l}_{2}} ( {θ}_{2} - {θ}_{1} ) )\end{matrix} ] \qquad ( 8 )$$

In the present embodiment, a linearized model near zero degrees that does not consider large deformation is obtained. From the above-described equations (4) and (5), the following equation (9) is obtained for the first bending section 171.

$${x}_{g 10} = {lim}_{{θ}_{1} \to 0} {x}_{g 1} = \frac{{l}_{g 1}^{2} {θ}_{1}}{2 {l}_{1}} , {z}_{g 10} = {lim}_{{θ}_{1} \to 0} {z}_{g 1} = {l}_{g 1} \qquad ( 9 )$$

Further, for the second bending section 172, based on the above-described equation (8), first, the first bending section 171 is approximated near zero degrees, and the following equation (10) is obtained.

$$[ \begin{matrix}{lim}_{{θ}_{1} \to 0} {x}_{g 2} \\ {lim}_{{θ}_{1} \to 0} {z}_{g 2}\end{matrix} ] = [ \begin{matrix}\frac{{l}_{1} {θ}_{1}}{2} \\ {l}_{1}\end{matrix} ] + [ \begin{matrix}1 & {θ}_{1} \\ - {θ}_{1} & 1\end{matrix} ] [ \begin{matrix}\frac{{l}_{2}}{{θ}_{2}} ( 1 - cos ( \frac{{l}_{g 2}}{{l}_{2}} {θ}_{2} ) ) \\ \frac{{l}_{2}}{{θ}_{2}} sin ( \frac{{l}_{g 2}}{{l}_{2}} {θ}_{2} )\end{matrix} ] \qquad ( 10 )$$

Next, the second bending section 172 is approximated near zero degrees, and the following equation (11) is obtained.

$$[ \begin{matrix}{lim}_{{θ}_{1} , {θ}_{2} \to 0} {x}_{g 2} \\ {lim}_{{θ}_{1} , {θ}_{2} \to 0} {z}_{g 2}\end{matrix} ] = [ \begin{matrix}\frac{{l}_{1} {θ}_{1}}{2} \\ {l}_{1}\end{matrix} ] + [ \begin{matrix}1 & {θ}_{1} \\ - {θ}_{1} & 1\end{matrix} ] [ \begin{matrix}\frac{{l}_{g 2}^{2} {θ}_{2}}{2 {l}_{2}} \\ {l}_{g 2}\end{matrix} ] \qquad ( 11 )$$

Accordingly, for the second bending section 172, the following equations (12) and (13) are obtained.

$${x}_{g 20} = {lim}_{{θ}_{1} , {θ}_{2} \to 0} {x}_{g 2} = \frac{{l}_{1} {θ}_{1}}{2} + \frac{{l}_{g 2}^{2} {θ}_{2}}{2 {l}_{2}} + {θ}_{1} {l}_{g 2} \qquad ( 12 )$$

$${z}_{g 20} = {lim}_{{θ}_{1} , {θ}_{2} \to 0} {z}_{g 2} = {l}_{1} - {θ}_{1} \frac{{l}_{g 2}^{2} {θ}_{2}}{2 {l}_{2}} + {l}_{g 2} \qquad ( 13 )$$

Here, a kinetic energy Tₐ₀ of the continuum portion near zero degrees is expressed by the following equation (14).

$${T}_{α0} = \frac{{m}_{s 1}}{2 {l}_{1}} \{ {\int }_{0}^{l} ( {\dot{x}}_{g 10}^{2} + {\dot{z}}_{g 10}^{2} ) {dl}_{g 1} \} + \frac{{m}_{s 2}}{2 {l}_{2}} \{ {\int }_{0}^{l} ( {\dot{x}}_{g 20}^{2} + {\dot{z}}_{g 20}^{2} ) {dl}_{g 2} \} \qquad ( 14 )$$

Further, a potential energy Uₐ of the continuum portion is expressed by the following equation (15).

$${U}_{a} = \frac{1}{2} {k}_{b 1} {θ}_{1}^{2} + \frac{1}{2} {{k}_{b 2} ( {θ}_{2} - {θ}_{1} )}^{2} \qquad ( 15 )$$

Then, a kinetic energy Tw and a potential energy U_{w} of the wire and the wire holding pipe are respectively expressed by the following equations (16) and (17) because driving amounts of the wires are lₚ₁=r₁θ₁ and lₚ₂=r₂θ₂.

$${T}_{w} = \frac{1}{2} {m}_{w 1} {\dot{z}}_{w 1}^{2} + \frac{1}{2} {m}_{p 1} {\dot{z}}_{p 1}^{2} + \frac{1}{2} {m}_{w 2} {\dot{z}}_{w 2}^{2} + \frac{1}{2} {m}_{p 2} {\dot{z}}_{p 2}^{2} \qquad ( 16 )$$

$$\begin{matrix}{U}_{w} = \frac{1}{2} {{k}_{w 1} ( {r}_{1} {θ}_{1} - {z}_{w 1} )}^{2} + \frac{1}{2} {{k}_{w 31} ( {z}_{w 1} - {z}_{p 1} )}^{2} \\ + \frac{1}{2} {{k}_{w 2} ( {r}_{2} {θ}_{2} - {z}_{w 2} )}^{2} + \frac{1}{2} {{k}_{w 32} ( {z}_{w 2} - {z}_{p 2} )}^{2}\end{matrix} \qquad ( 17 )$$

Derivation of equations of motion from Lagrange's equation expressed by the following equation (18) is considered.

$$\frac{d}{dt} ( \frac{\partial T}{\partial {\dot{q}}_{i}} ) - \frac{\partial T}{\partial {q}_{i}} + \frac{\partial U}{\partial {q}_{i}} = {Q}_{i} i = 1 , 2 \qquad ( 18 )$$

$$T = {T}_{a 0} + {T}_{w} , U = {U}_{a} + {U}_{w}$$

$$q = {[ {q}_{1} , … , {q}_{6} ]}^{T} = {[ {z}_{p 1} , {z}_{w 1} , {θ}_{1} , {z}_{p 2} , {z}_{w 2} , {θ}_{2} ]}^{T}$$

$$Q = {[ {Q}_{1} , … , {Q}_{6} ]}^{T} = {[ 0 , 0 , 0 , 0 , 0 , 0 ]}^{T}$$

As a result, the equations of motion are expressed by the following equation (19).

$$( 19 )$$

$$\text{?} = [ \begin{matrix}\text{?} & 0 & 0 & 0 & 0 & 0 \\ 0 & \text{?} & 0 & 0 & 0 & 0 \\ 0 & 0 & \text{?} & 0 & 0 & \text{?} \\ 0 & 0 & 0 & \text{?} & 0 & 0 \\ 0 & 0 & 0 & 0 & \text{?} & 0 \\ 0 & 0 & \text{?} & 0 & 0 & \text{?}\end{matrix} ] [ \text{?} ] + [ \begin{matrix}\text{?} & \text{?} & 0 & 0 & 0 & 0 \\ \text{?} & \text{?} & \text{?} & 0 & 0 & 0 \\ 0 & \text{?} & \text{?} & 0 & 0 & \text{?} \\ 0 & 0 & 0 & \text{?} & \text{?} & 0 \\ 0 & 0 & 0 & \text{?} & \text{?} & \text{?} \\ 0 & 0 & \text{?} & 0 & \text{?} & \text{?}\end{matrix} ] [ \text{?} ] + [ \begin{matrix}\text{?} & \text{?} & 0 & 0 & 0 & 0 \\ \text{?} & \text{?} & \text{?} & 0 & 0 & 0 \\ 0 & \text{?} & \text{?} & 0 & 0 & 0 \\ 0 & 0 & 0 & \text{?} & \text{?} & 0 \\ 0 & 0 & 0 & \text{?} & \text{?} & \text{?} \\ 0 & 0 & \text{?} & 0 & \text{?} & \text{?}\end{matrix} ] [ \text{?} ] = [ \text{?} ]$$

$$\text{?} \text{indicates text missing or illegible when filed}$$

### [2] Model Including Rotary Motor, Rotary-to-Linear Conversion Mechanism, and Wire Holding Mechanism

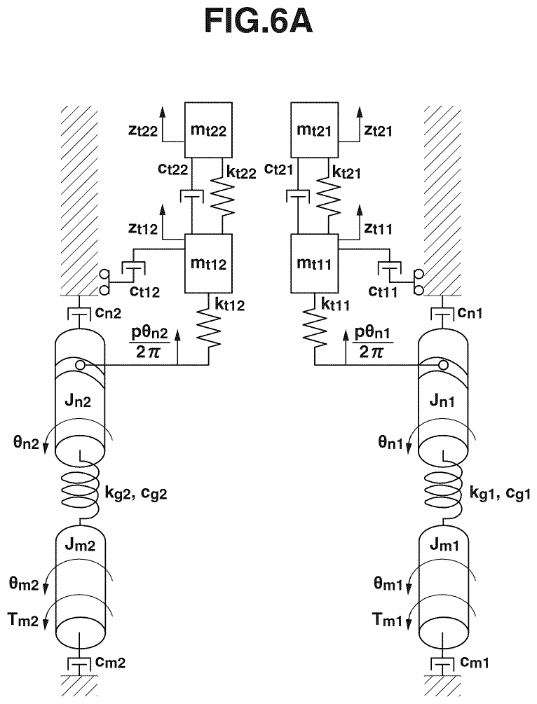

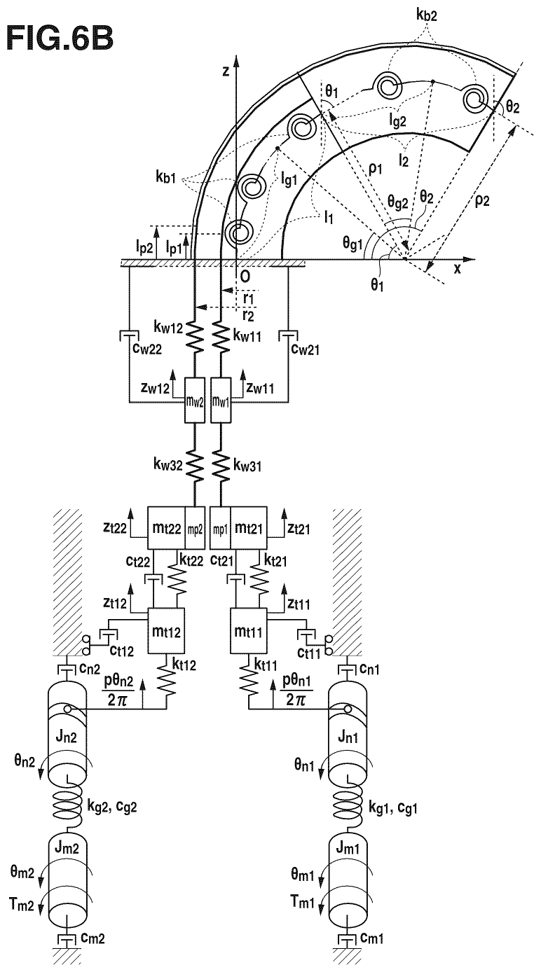

FIG. 6A is a diagram illustrating an example of a model including a rotary motor, a rotary-to-linear conversion mechanism, and a wire holding mechanism in the continuum robot 100 according to the third embodiment. The wire holding mechanism has a function of detecting tension of a wire. A wire holding mechanism base is provided between the wire holding mechanism and the actuator, the wire holding mechanism base is connected to the actuator, and the wire holding mechanism base and the wire holding mechanism are connected by a spring. The tension of the wire is detected by detecting displacement of the spring. Reference symbols are defined as follows. Note that a subscript “n” indicating an n-th bending section is attached to ends of all the following reference symbols, but the subscript “n” is omitted in the text.
Jₘ: inertia of the motor
θₘ: rotational angle of the motor
Tₘ: torque command of the motor
cₘ: damping coefficient of a motor shaft
k_{g}, c_{g}: spring constant and damping coefficient of the coupling
Jₙ: inertia of a driving shaft
θₙ: rotational angle of the driving shaft
cₙ: damping coefficient of the driving shaft
p: screw pitch of the driving shaft
R: conversion coefficient of the rotary-to-linear conversion mechanism, i.e., equivalent speed increasing ratio
mₜ₁: mass of the wire holding mechanism base
kₜ₁: spring constant in the z direction of the driving shaft
cₜ₁: damping coefficient of a linear slider of the wire holding mechanism base
zₜ₁: displacement of the wire holding mechanism base
mₜ₂: mass of the wire holding mechanism
kₜ₂: spring constants of a tension detection mechanism
zₜ₂: displacement of the wire holding mechanism

The equations of motion are expressed by the following equations (20) to (23).

$${J}_{m} {\ddot{θ}}_{m} = {T}_{m} + {k}_{g} ( {θ}_{n} - {θ}_{m} ) + {c}_{g} ( {\dot{θ}}_{n} - {\dot{θ}}_{m} ) - {c}_{m} {\dot{θ}}_{m} \qquad ( 20 )$$

$${J}_{n} {\ddot{θ}}_{n} = - {k}_{g} ( {θ}_{n} - {θ}_{m} ) - {c}_{g} ( {\dot{θ}}_{n} - {\dot{θ}}_{m} ) + R \text{?} ( \text{?} - R {θ}_{n} ) - {c}_{n} {\dot{θ}}_{n} \qquad ( 21 )$$

$$\text{?} = - \text{?} ( \text{?} - R {θ}_{n} ) + {k}_{t 2} ( {z}_{t 2} - {\dot{z}}_{t 1} ) - \text{?} + {c}_{t 2} ( {\dot{z}}_{t 2} - {\dot{z}}_{t 1} ) \qquad ( 22 )$$

$$\text{?} = - \text{?} ( {z}_{t 2} - \text{?} ) - \text{?} ( {\dot{z}}_{t 2} - {\dot{z}}_{t 1} ) \qquad ( 23 )$$

$$\text{?} \text{indicates text missing or illegible when filed}$$

Here, in the above-described equations (21) and (22), R=p/2π is assumed. Further, where qₗ=[θₘ, θₙ, zₜ₁, zₜ₂]^{T}, the matrix representation is given by the following equation (24).

$$\text{?} = [ \begin{matrix}\text{?} & & & 0 \\ & \text{?} & & \\ & & \text{?} & \\ 0 & & & \text{?}\end{matrix} ] [ \text{?} ] + [ \begin{matrix}\text{?} & \text{?} & 0 & 0 \\ \text{?} & \text{?} & \text{?} & 0 \\ 0 & \text{?} & \text{?} & \text{?} \\ 0 & 0 & \text{?} & \text{?}\end{matrix} ] [ \text{?} ] + [ \begin{matrix}\text{?} & \text{?} & 0 & 0 \\ \text{?} & \text{?} & 0 & 0 \\ 0 & 0 & \text{?} & \text{?} \\ 0 & 0 & \text{?} & \text{?}\end{matrix} ] [ \text{?} ] = [ \begin{matrix}\text{?} & 0 \\ 0 & 0 \\ 0 & 0 \\ 0 & \text{?}\end{matrix} ] [ \text{?} ] \qquad ( 24 )$$

$$\text{?} \text{indicates text missing or illegible when filed}$$

Using the equation (24), the model including the rotary motor, the rotary-to-linear conversion mechanism, and the wire holding mechanism for driving the n-th bending section can be expressed by the following equation (25).

$$\text{?} + \text{?} + \text{?} = \text{?} \text{?} = {[ \text{?} ]}^{T} \text{?} \text{?} = {[ \text{?} ]}^{T} \qquad ( 25 )$$

$$\text{?} \text{indicates text missing or illegible when filed}$$

### [3] Configuration of Expanded System

FIG. 6B is a diagram illustrating a configuration example in which the dynamic model of the continuum portion illustrated in FIG. 5 and the model including the rotary motor, the rotary-to-linear conversion mechanism, and the wire holding mechanism illustrated in FIG. 6A are joined in the continuum robot 100 according to the third embodiment. For the first bending section 171, the displacement zₜ₂₁ of the wire holding mechanism (wire holding unit 212) and a displacement zₚ₁ of the wire holding pipe are identical to each other. For the second bending section 172, the displacement zₜ₂₂ of the wire holding mechanism (wire holding unit 222) and a displacement zₚ₂ of the wire holding pipe are identical to each other. Therefore, when an expanded system is configured based on the above-described equations (19) and (24), the equations of motion are expressed by the following equation (26).

$${M}_{g} {\ddot{q}}_{g} + {K}_{g} {q}_{g} + {C}_{g} {\dot{q}}_{g} = [ \begin{matrix}{H}_{1} & 0 \\ 0 & {H}_{2}\end{matrix} ] [ \begin{matrix}{u}_{υ 1} \\ {u}_{υ 2}\end{matrix} ] = {H}_{m} u \qquad ( 26 )$$

$${q}_{g} = {[ {q}_{l 1}^{T} , {z}_{w 1} , {θ}_{1} , {q}_{l 2}^{T} , {z}_{w 2} , {θ}_{2} ]}^{T}$$

Next, state equations are expressed by the following equations (27) and (28).

$$\text{?} = {A}_{g} {x}_{g} + {B}_{g} u \qquad ( 27 )$$

$$( 28 )$$

$${x}_{g} = {[ {θ}_{m 1} , {θ}_{n 1} , \text{?} , {θ}_{1} , \text{?} , {θ}_{n 2} , \text{?} , {z}_{t 22} , \text{?} , {θ}_{2} , {\dot{θ}}_{m 1} , {\dot{θ}}_{n 1} , \text{?} , {\dot{θ}}_{1} , {\dot{θ}}_{m 2} , {\dot{θ}}_{n 2} , \text{?} , {z}_{t 22} , \text{?} , {\dot{θ}}_{2} ]}^{T}$$

$$\text{?} \text{indicates text missing or illegible when filed}$$

In the present embodiment, a displacement zₜ₁ₙ of the wire holding mechanism base and a displacement zₜ₂ₙ of the wire holding mechanism can be measured. A force Fₙ that is generated at a spring constant kₜ₂ₙ and acts on the wire holding mechanism (hereinafter referred to as a “generated force”) and the displacement zₜ₂ₙ of the wire holding mechanism are taken as observed quantities. Here, the generated force Fₙ is expressed by the following equation (29).

$${F}_{n} = - {k}_{t 2 n} ( {z}_{t 2 n} - {z}_{t 1 n} ) \qquad ( 29 )$$

At this time, an observed quantity y_{g} is expressed by an output equation expressed by the following equation (30).

$$\begin{matrix}\text{?} = {[ \begin{matrix}\text{?} & \text{?} & \text{?}\end{matrix} ]}^{T} \\ = \text{?}\end{matrix} \qquad ( 30 )$$

$$\text{?} \text{indicates text missing or illegible when filed}$$

### [4] Simulation

By using the model of the continuum robot 100 expressed by the equations (27) and (30), variation of the model relative to variation of damping coefficients c_{w21} and c_{w22} of the coupling control dampers is simulated.

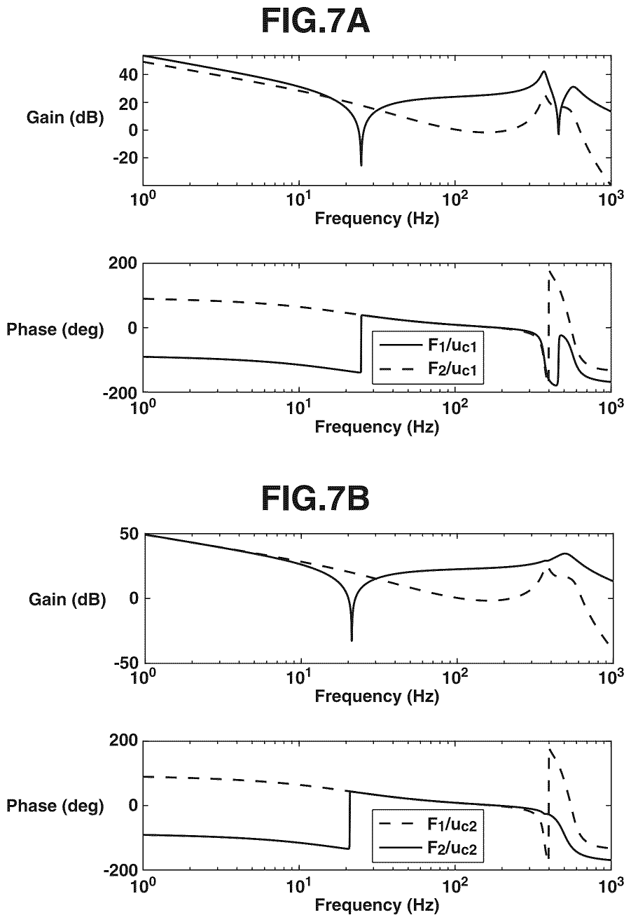

FIGS. 7A and 7B are diagrams illustrating an example of a frequency response when c_{w21}=c_{w22}=0 Ns/m is set in the continuum robot 100 according to the third embodiment. More specifically, in FIG. 7A, with a solid line, a transfer function from a control input u_{c1} for the first bending section 171 to a generated force F₁ of the wire in the first bending section 171 is illustrated, and with a dashed line, a transfer function to a generated force F₂ of the wire in the second bending section 172 is illustrated. In FIG. 7B, with a solid line, a transfer function from a control input u_{c2} for the second bending section 172 to the generated force F₂ of the wire in the second bending section 172 is illustrated, and with a dashed line, a transfer function to the generated force F₁ of the wire in the first bending section 171 is illustrated. In other words, in each of FIGS. 7A and 7B, a frequency response by coupling between the bending sections is illustrated with a dashed line, and a phase exceeds 180 degrees near 400 Hz. For example, it can be seen that, when a proportional (P) controller is used for the force control unit K_{F} and a gain is increased, the control system becomes unstable due to the coupling between the bending sections.

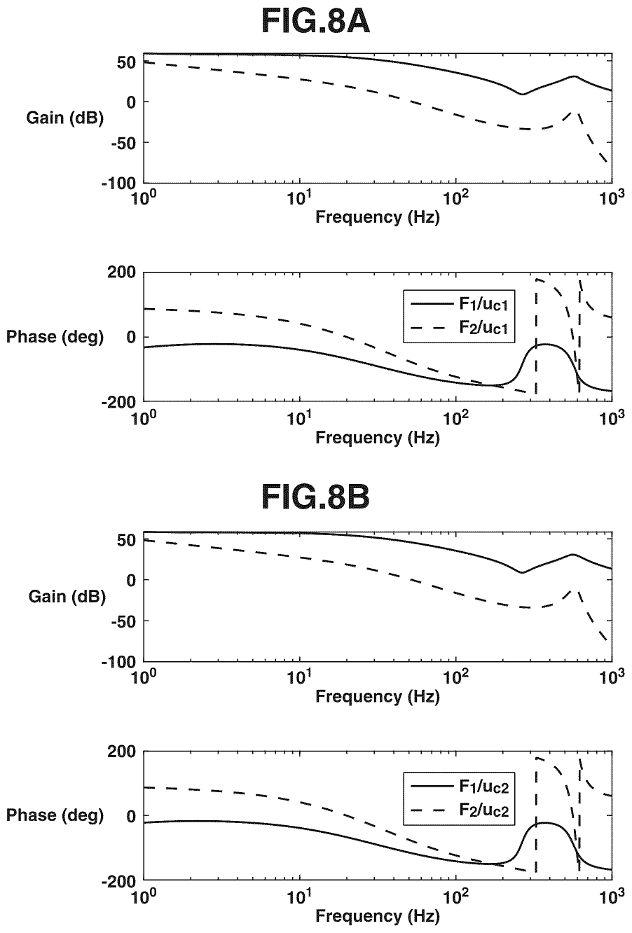

FIGS. 8A and 8B are diagrams illustrating an example of a frequency response when c_{w21}=c_{w22}=150 Ns/m is set in the continuum robot 100 according to the third embodiment. More specifically, in FIG. 8A, with a solid line, a transfer function from the control input u_{c1} for the first bending section 171 to the generated force F₁ of the wire in the first bending section 171 is illustrated, and with a dashed line, a transfer function to the generated force F₂ of the wire in the second bending section 172 is illustrated. In FIG. 8B, with a solid line, a transfer function from the control input u_{c2} for the second bending section 172 to the generated force F₂ of the wire in the second bending section 172 is illustrated, and with a dashed line, a transfer function to the generated force F₁ of the wire in the first bending section 171 is illustrated. It can be seen from FIGS. 8A and 8B that gain characteristics due to coupling between the bending sections are sufficiently lower than direct gain characteristics. Accordingly, it can be seen that it is sufficient to design the control system by considering only the frequency response. Furthermore, it can be seen that the direct frequency responses in FIGS. 8A and 8B are similar to each other, and in a case where distributed control is performed using the same controller, there is no significant difference in bending drive control characteristics.

### Fourth Embodiment

Next, a fourth embodiment is described. In the following description of the fourth embodiment, description of matters common to the above-described first to third embodiments is omitted, and matters different from the above-described first to third embodiments are described.

In the above-described third embodiment, it has been described that a response is compared between the case where the values of the coupling control dampers 813 and 823 are set to zero and the case where the values of the coupling control dampers 813 and 823 are set to large values, and providing the large values reduces the coupling between the bending sections. Accordingly, it has been described that it is possible to stably design the distributed control in which the control unit (control system) is designed for a single bending section without considering coupling and is arranged in each bending section. In the fourth embodiment, it is described that by setting appropriate values for the coupling control dampers 813 and 823, it is possible to improve control performance of the entire control system including coupling.

### [1] Design of Control System

In the fourth embodiment, a control system that provides high backdrivability while enabling positioning is designed for a continuum robot 100 by using the dual-loop control system in which the inner loop control system performs force control and the outer loop control system performs position control as illustrated in FIG. 2B. Here, Pₙ indicates the expanded system expressed by the equations (27) and (30). Further, ref_{θn} indicates a target command for the bending angle in the n-th bending section, ref_{zn} indicates a target displacement of the wire holding mechanism in the n-th bending section, and ref_{F} indicates a target value of the generated force F. Relationship between the driving amount lₚ of the wire and the bending angle θ is given by the following equation (31).

$${l}_{p} = {r}_{1} θ \qquad ( 31 )$$

In the fourth embodiment, elongation of the wire is assumed to be small and is not considered in derivation of kinematics. Accordingly, the target displacement ref_{zn} is given by the following equation (32).

$$r e {f}_{zn} = {r}_{1} \cdot {ref}_{θ n} \qquad ( 32 )$$

In the inner loop control system including the expanded system Pₙ and the force control unit K_{F}, an error is calculated by calculating a difference between the target value ref_{F} of the generated force and the generated force F, and the force control unit K_{F} outputs a motor torque Tₘ as a control input for compensating the error. The feedback loop control system is equivalent to compensating for equivalent inertia of the rotary-to-linear conversion mechanism, thereby making it possible to improve the backdrivability of the continuum robot 100. In the fourth embodiment, a proportional-integral (PI) control system expressed by the following equation (33) is used for the force control unit K_{F}.

$${K}_{F} ( s ) = {K}_{p} ( 1 + \frac{{K}_{i}}{s} ) , {K}_{i} = 2 π {F}_{zi} \qquad ( 33 )$$

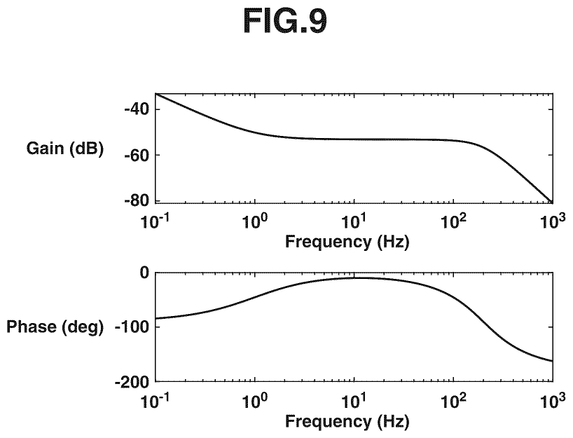

Here, in the equation (33), F_{zi} is a zero-cross frequency of an integral controller. Further, for stabilization against a higher-order mode, a second-order lowpass filter having a corner frequency of 30 Hz is joined to the PI control system. FIG. 9 is a Bode diagram of the force control unit K_{F} in a case where a gain Kₚ is set to 2.2·10⁻³, the zero-cross frequency F_{zi} is set to 1.0, and a control bandwidth is set to approximately 10 Hz in the system 10 for controlling the continuum robot according to the fourth embodiment.

Next, a state-space model of the continuum robot 100 expressed by the equations (27) and (30) is denoted by P, and a closed-loop system G_{cl} expressed by the following equation (34) is determined.

$${G}_{cl} = \frac{{PK}_{PI}}{I + {PK}_{PI}} \qquad ( 34 )$$

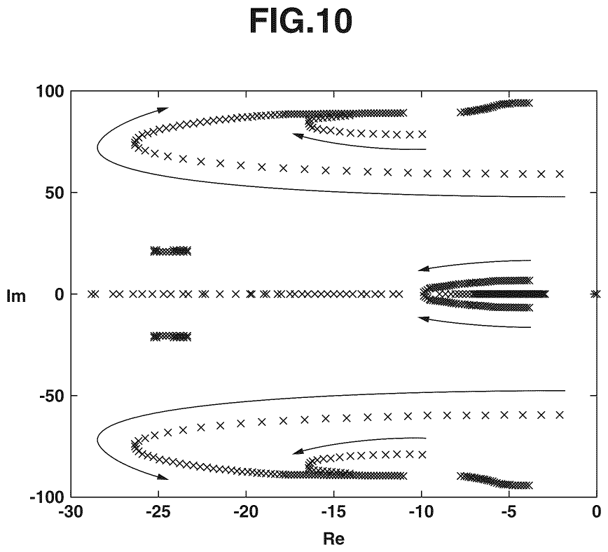

Then, eigenvalues of the closed-loop system are calculated while the damping coefficient c_{w21} of the coupling control damper 813 and the damping coefficient c_{w22} of the coupling control damper 823 are varied by 1 Ns/m from 0 Ns/m to 150 Ns/m. FIG. 10 is a diagram obtained by plotting variation of the eigenvalues of the closed-loop system in the system 10 for controlling the continuum robot according to the fourth embodiment. The eigenvalues of the system become more stable as the real part of a complex number decreases; however, at some poles, the real part of the complex number does not monotonically decrease with an increase in the damping coefficients of the coupling control damper. Therefore, it can be seen that there is an optimal value for the damping coefficients of the coupling control damper to stabilize the closed-loop system. In the fourth embodiment, attention is focused on a root locus that draws a maximum arc as illustrated in FIG. 10, and the damping coefficient of the coupling control damper that has a pole at which the value of the real part of the locus is minimum is regarded as a quasi-optimal damping coefficient.

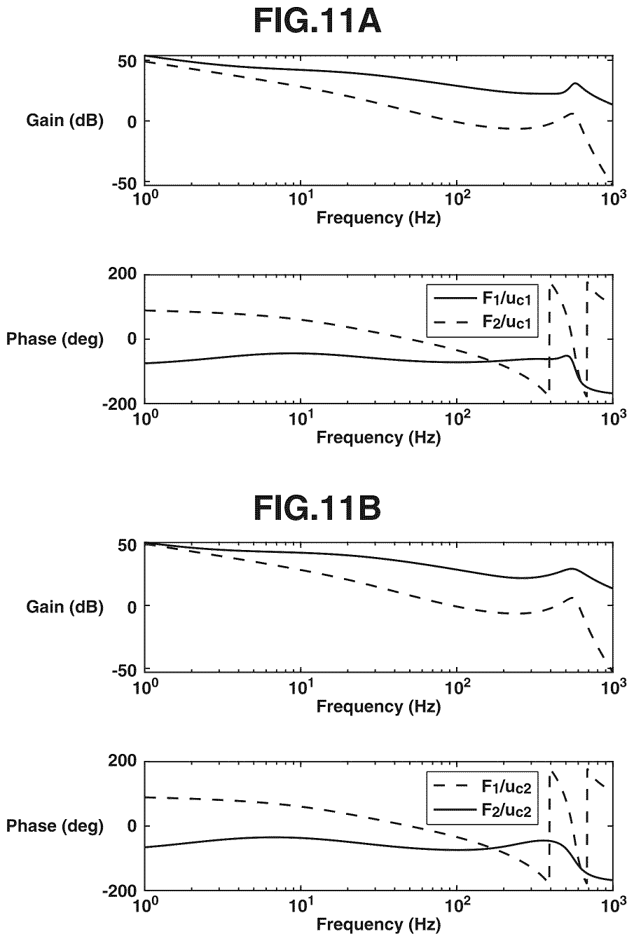

FIGS. 11A and 11B are diagrams illustrating an example of a frequency response in a continuum robot 100 according to the fourth embodiment, where the quasi-optimal damping coefficients of the coupling control dampers are set to c_{w21}=c_{w22}=25 Ns/m in the system 10 for controlling the continuum robot. More specifically, in FIG. 11A, with a solid line, a transfer function from the control input u_{c1} for the first bending section 171 to the generated force F₁ of the wire in the first bending section 171 is illustrated, and with a dashed line, a transfer function to the generated force F₂ of the wire in the second bending section 172 is illustrated. In FIG. 11B, with a solid line, a transfer function from the control input u_{c2} for the second bending section 172 to the generated force F₂ of the wire in the second bending section 172 is illustrated, and with a dashed line, a transfer function to the generated force F₁ of the wire in the first bending section 171 is illustrated.

It can be seen from FIGS. 11A and 11B that gain characteristics due to coupling between the bending sections are sufficiently lower than direct gain characteristics at 100 Hz or more at which the phase is close to 180 degrees. Further, it can be seen that the direct frequency responses in FIGS. 11A and 11B are similar to each other, and in a case where distributed control is performed using the same controller, there is no significant difference in bending drive control characteristics.

In the fourth embodiment, a PID control system expressed by the following equation (35) is used for the position control unit K_{SV}.

$${K}_{SV} ( s ) = {K}_{p} ( 1 + \frac{{K}_{i}}{s} + \frac{s}{{K}_{d}} ) , {K}_{i} = 2 π {F}_{zi} , {K}_{d} = 2 π {F}_{zd} \qquad ( 35 )$$

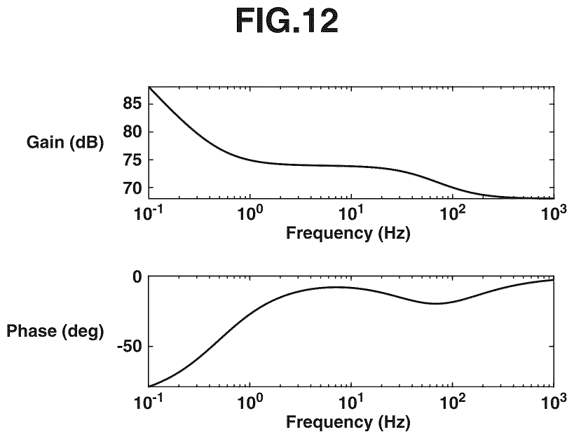

Here, in the equation (35), F_{zi} is a zero-cross frequency of the integral controller, and F_{zd} is a zero-cross frequency of a derivative controller. In the fourth embodiment, an open-loop transfer function G_{cl}K_{SV} is derived using the closed-loop transfer function (closed-loop system) G_{cl} of the expanded system Pₙ and the force control unit K_{F}, and is designed so that a gain margin and a phase margin are sufficient from the response. Further, in the fourth embodiment, a first-order lowpass filter having a corner frequency of 50 Hz is joined to the PID control system. FIG. 12 is a Bode diagram of the position control unit K_{SV} in a case where the gain Kₚ is set to 5·10³, the zero-cross frequency F_{zi} is set to 0.5, and the zero-cross frequency F_{zd} is set to 100 in the system 10 for controlling the continuum robot according to the fourth embodiment.

### [2] Simulation

Simulation using a model in which the damping coefficients of the coupling control dampers determined in “[1] Design of Control System” are set to c_{w21}=c_{w22}=25 Ns/m (hereinafter referred to as “quasi-optimal damping model”) is performed. As a comparison, simulation using models in which the damping coefficients of the coupling control dampers determined in the third embodiment are set to c_{w21}=c_{w22}=0 Ns/m and c_{w21}=c_{w22}=150 Ns/m (hereinafter respectively referred to as “low damping model” and “high damping model”) is performed. The simulation uses the control system illustrated in FIGS. 2A and 2B that is used in the first embodiment, and the coupling control unit K_{D} for controlling the coupling control actuator is set to zero, while the coupling control dampers are used as described in the third embodiment. Further, the simulation includes the coupling control damper in a dynamic model P of the continuum robot 100.

For both the first bending section 171 and the second bending section 172, the PI control system expressed by the equation (33) is used for the force control unit K_{F}. However, since the gain is different between the quasi-optimal model and the high damping model, the gain Kₚ of the PI control system is adjusted so as to realize an identical servo bandwidth. In addition, for the low damping model, response diverges in the servo bandwidth identical to that of the quasi-optimal model. Thus, the gain is adjusted so that the control input does not become oscillatory.

For the first bending section 171, the PID control system expressed by the equation (35) is used for the position control unit K_{SV}. Further, a reference command ref_{z1} is given to the displacement zₜ₂₁ of the wire holding mechanism so that the bending angle becomes 45 degrees after 0.5 seconds from start of the simulation.

For the second bending section 172, a position control gain is set to zero. Further, a control problem is defined such that positioning is performed through coupling with the first bending section 171. This is because, if a position control system is used for the second bending section 172, an effect of optimizing the designed coupling control damper may not become clear. When the damping of the coupling control damper is appropriate, the second bending section 172 promptly backdrives and follows the angle of the first bending section 171. This makes it possible to evaluate performance of the coupling control damper based on time history.

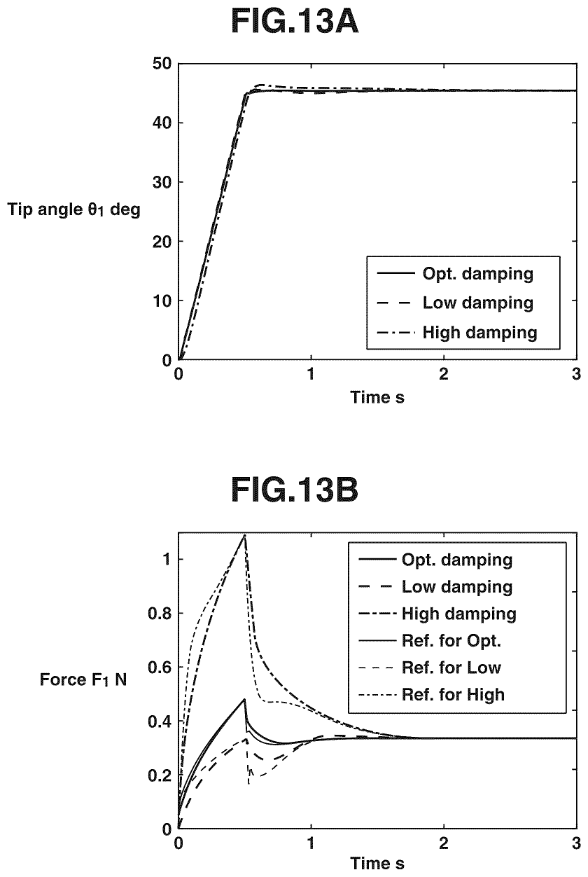

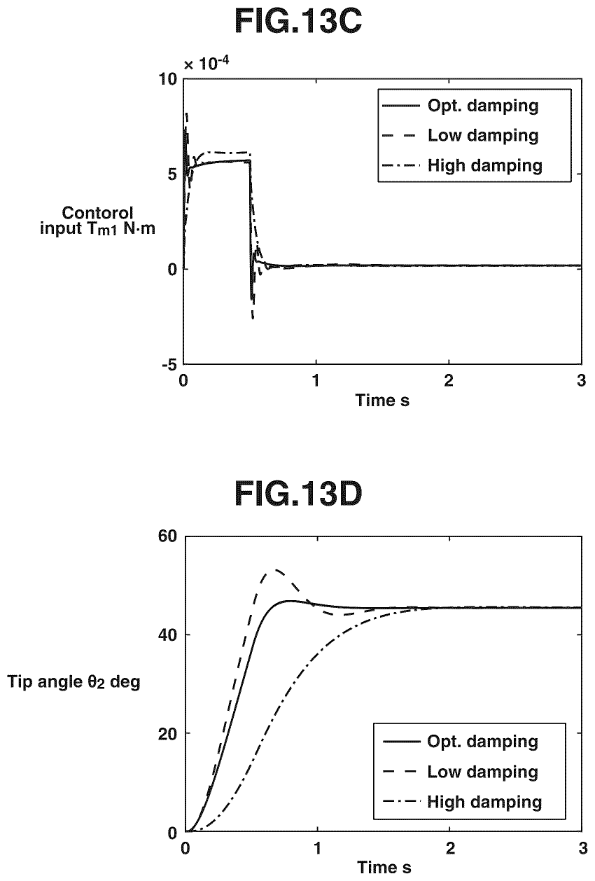

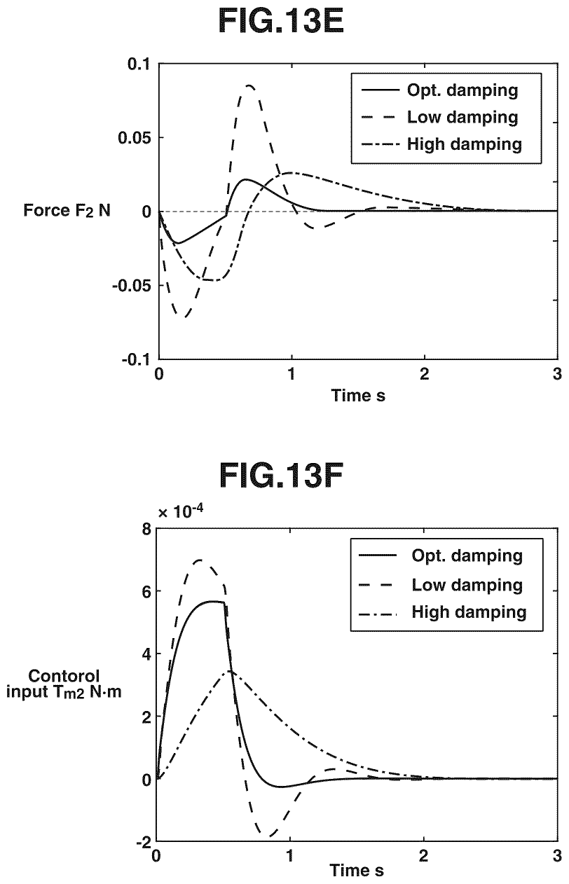

FIGS. 13A to 13F are diagrams each illustrating simulation responses in the system 10 for controlling the continuum robot according to the fourth embodiment, where the quasi-optimal model is illustrated with a solid line, the low damping model is illustrated with a dashed line, and the high damping model is illustrated with a dash-dot line. FIG. 13A illustrates responses with regard to an angle of the distal end of the first bending section 171. The first bending section 171 is controlled by a position servo system, and accordingly, there is no significant difference in the responses. FIG. 13B illustrates generated forces for the wire and reference values thereof for the first bending section 171 with thick and thin lines, respectively. In FIG. 13B, because the high damping model has a strong damping force of the coupling control damper, a large tension of the wire is generated. FIG. 13C illustrates the control inputs for the first bending section 171. In FIG. 13C, it can be seen that the control input for the low damping model is slightly oscillatory. FIG. 13D illustrates responses with regard to an angle of the distal end of the second bending section 172. In FIG. 13D, in the quasi-optimal model, the response is coupled with the motion of the first bending section 171, and the angle promptly converges to 45 degrees. In FIG. 13D, however, the response of the low damping model becomes oscillatory, whereas the response of the high damping model to converge to 45 degrees is delayed because damping of the coupling control damper is strong. FIG. 13E illustrates the generated force of the wire in the second bending section 172. In FIG. 13E, a reference value of the force in all cases is zero in all cases because the positioning loop is not enabled for the second bending section 172. Further, it can be seen that in the response of the quasi-optimal model, the generated force promptly converges to zero, and high backdrivability is obtained. However, in the low damping model, the generated force is oscillatory, and in the high damping model, convergence of the generated force is delayed because the damping by the coupling control damper is strong. FIG. 13F illustrates the control inputs for the second bending section 172. In the high damping model, convergence of the control input is delayed because the damping is large.

As described above, it can be seen that, by determining the quasi-optimal values of the coupling control dampers using the dynamic model, the coupling between the bending sections becomes appropriate, and neither oscillatory coupling nor response delay due to the coupling occurs.

### OTHER EMBODIMENTS

The present disclosure can be realized by supplying a program realizing one or more functions of the above-described embodiments to a system or an apparatus through a network or a storage medium, and causing one or more processors in a computer of the system or the apparatus to read out and execute the program. Further, the present disclosure can be realized by a circuit (e.g., application specific integrated circuit (ASIC)) realizing one or more functions. The program and a computer-readable storage medium storing the program are included in the present disclosure.

The above-described embodiments of the present disclosure are merely examples of specific implementations of the present disclosure, and should not be construed as limiting the technical scope of the present disclosure. Thus, the present disclosure can be implemented in various forms without departing from the technical idea or the main features of the present disclosure.

The embodiments of the present disclosure includes the following configurations and methods.

### [Configuration 1]

A system for controlling a continuum robot, the continuum robot including a bendable portion and a wire driving unit, the bendable portion including a plurality of bending sections each driven by a wire, the wire driving unit being configured to drive the wire for each of the plurality of bending sections, the system comprising:
a control unit configured to control operation of the wire driving unit for each of the plurality of bending sections; and
a suppression unit configured to, when the wire driving unit for one of the plurality of bending sections is operated, suppress variation in tension of the wires for other bending sections.

### [Configuration 2]

The system for controlling the continuum robot according to configuration 1,
wherein the continuum robot further includes a tension detection unit interposed between the wire and the wire driving unit and configured to detect the tension of the wire for each of the plurality of bending sections, and
wherein the suppression unit includes:
a second wire driving unit configured to drive the wire without interposing the tension detection unit; and
a second control unit configured to control operation of the second wire driving unit based on displacement of a displacement detection unit provided to either the wire extending between the corresponding bending section and the tension detection unit or a wire connection unit connecting the wire and the tension detection unit.

### [Configuration 3]

The system for controlling the continuum robot according to configuration 2, wherein the second wire driving unit is connected to the wire connection unit.

### [Configuration 4]

The system for controlling the continuum robot according to configuration 2, wherein the second wire driving unit is connected to the wire.

### [Configuration 5]

The system for controlling the continuum robot according to any one of configurations 2 to 4, wherein the second control unit includes a control system using a differentiator.

### [Configuration 6]

The system for controlling the continuum robot according to configuration 1, wherein the suppression unit is a damper connected to a wire connection unit connecting the wire and the wire driving unit.

### [Configuration 7]

The system for controlling the continuum robot according to configuration 1, wherein the suppression unit is a damper connected to the wire.

### [Configuration 8]

The system for controlling the continuum robot according to configuration 6 or 7, wherein a damping coefficient of the damper is set using a dynamic model of the continuum robot. [Method 1]

A method for controlling a continuum robot, the continuum robot including a bendable portion and a wire driving unit, the bendable portion including a plurality of bending sections each driven by a wire, the wire driving unit being configured to drive the wire for each of the plurality of bending sections, the method comprising:
controlling operation of the wire driving unit for each of the plurality of bending sections by using a control unit provided for each of the plurality of bending sections; and
suppressing, when the wire driving unit for one of the plurality of bending sections is operated, variation in tension of the wires for other bending sections by using a suppression unit.

The present disclosure is not limited to the above embodiments and can be modified and varied in various ways without departing from the spirit and scope of the present disclosure. Accordingly, the following claims are appended to publicly indicate the scope of the present disclosure.

According to the present disclosure, it is possible to suppress a decrease in stability of the control system of the continuum robot.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A system for controlling a continuum robot, the continuum robot including a bendable portion and a wire driving unit,
the bendable portion including
a first bending section driven by a first wire, and
a second bending section driven by a second wire,
the wire driving unit being configured to drive the first wire and the second wire, the system comprising:
a control unit configured to control operation of the wire driving unit for the first bending section and for the second bending section; and
a suppression unit configured to, when the wire driving unit is operated to drive the first wire, suppress variation in tension of the second wire and when the wire driving unit is operated to drive the second wire, suppress variation in tension of the first wire.

2. The system for controlling the continuum robot according to claim 1,
wherein the continuum robot further includes a tension detection unit interposed between the first wire and the wire driving unit and configured to detect the tension of the first wire, and
wherein the suppression unit includes:
a second wire driving unit configured to drive the first wire without interposing the tension detection unit; and
a second control unit configured to control operation of the second wire driving unit based on displacement of a displacement detection unit provided to either the first wire extending between the first bending section and the tension detection unit or a wire connection unit connecting the first wire and the tension detection unit.

3. The system for controlling the continuum robot according to claim 2, wherein the second wire driving unit is connected to the wire connection unit.

4. The system for controlling the continuum robot according to claim 2, wherein the second wire driving unit is connected to the first wire.

5. The system for controlling the continuum robot according to claim 2, wherein the second control unit includes a control system using a differentiator.

6. The system for controlling the continuum robot according to claim 1, wherein the suppression unit is a damper connected to a wire connection unit connecting the first wire and the wire driving unit.

7. The system for controlling the continuum robot according to claim 1, wherein the suppression unit is a damper connected to the first wire.

8. The system for controlling the continuum robot according to claim 6, wherein a damping coefficient of the damper is set using a dynamic model of the continuum robot.

9. The system for controlling the continuum robot according to claim 7, wherein a damping coefficient of the damper is set using a dynamic model of the continuum robot.

10. A method for controlling a continuum robot, the continuum robot including a bendable portion and a wire driving unit,
the bendable portion including a first bending section driven by a first wire and a second bending section driven by a second wire,
the wire driving unit being configured to drive the first wire and the second wire, the method comprising:
controlling operation of the wire driving unit for of the first bending section and the second bending section by using a control unit provided for the first bending section and for the second bending section; and
suppressing, when the wire driving unit for one of the first or second bending section is operated, variation in tension of the wires for second or first bending section, respectively, by using a suppression unit.

11. The system for controlling the continuum robot according to claim 1,
wherein the bendable portion incudes a third bending section driven by a third wire,
wherein the control unit is further configured to control operation of the wire driving unit for the third bending section; and
wherein the suppression unit is further configured to, when the wire driving unit for the third bending section is operated, suppress variation in tension of the first and second wires.

12. The system for controlling the continuum robot according to claim 2,
wherein the continuum robot further includes a second tension detection unit interposed between the second wire and the wire driving unit and configured to detect the tension of the second wire, and
wherein the suppression unit includes:
a third wire driving unit configured to drive the second wire without interposing the second tension detection unit; and
a third control unit configured to control operation of the third wire driving unit based on displacement of a second displacement detection unit provided to either the second wire extending between the second bending section and the second tension detection unit or a second wire connection unit connecting the second wire and the second tension detection unit.

13. The system for controlling the continuum robot according to claim 1,
wherein the wire driving unit comprises a first wire driving unit configured to drive the first wire and a second wire driving unit configured to drive the second wire.

14. The system for controlling the continuum robot according to claim 1, wherein the suppression unit comprises a first suppression unit configured to suppress variation in tension of the second wire and a second suppression unit configured to suppress variation in tension of the first wire
